(19) United States
(12) Patent Application Publication
(10) Pub. No.: US 2025/0042826 A1
(43) Pub. Date: Feb. 6, 2025

(54) METHOD OF DEOXYGENATION OF A HYDROCARBON IN THE PRESENCE OF METHANE-CONTAINING GAS ENVIRONMENT AND CATALYST STRUCTURE

(71) Applicant: Kara Technologies Inc., Calgary AB (CA)

(72) Inventors: Hua Song, Calgary (CA); Yimeng Li, Calgary (CA); Hao Xu, Calgary (CA); Zhaofei Li, Calgary (CA)

(21) Appl. No.: 18/918,489

(22) Filed: Oct. 17, 2024

Related U.S. Application Data
(63) Continuation of application No. PCT/IB2022/053945, filed on Apr. 28, 2022.

(51) Int. Cl. C07C 1/20; B01J 27/10; B01J 27/25; B01J 37/08

(52) U.S. Cl. CPC C07C 1/20; B01J 27/10; B01J 27/25; B01J 37/08; C07C 2521/06

(57) ABSTRACT

A method for deoxygenation of an oxygen-containing hydrocarbon, such as a bio-crude liquid, in the presence of a methane-containing gas and a catalyst structure, is described. The method results in a reduction of oxygen content within the hydrocarbon.

### CROSS-REFERENCE TO RELATED APPLICATION

The present application is a continuation of PCT International Application No. PCT/IB2022/053945, filed on Apr. 28, 2022 and entitled “METHOD OF DEOXYGENATION OF A HYDROCARBON IN THE PRESENCE OF METHANE-CONTAINING GAS ENVIRONMENT AND CATALYST STRUCTURE”, the disclosure of which is incorporated herein by reference in its entirety.

### FIELD

The invention relates to renewable bio-fuel processing.

### BACKGROUND

In modern society, most activities heavily rely on fossil fuels which provide more than three-quarters of the world's energy consumption. It has led to many international concerns including dwindling sources, environmental, economic, and political problems. Therefore, biofuels as an alternative renewable energy source have attracted attention and become widely accepted. Nowadays, biodiesel and bioethanol can be substituted for conventional fuels. Another promising feedstock for the production of engine fuels is the so-called bio-crude, the liquid product of biomass flash pyrolysis. However, this bio-crude cannot be directly used as a fuel for spark engines due to its poor operational characteristics which are mainly reflected in the high oxygen content. Hence, further treatments for bio-crude are needed to make it suitable for engine uses.

The traditional deoxygenation process for bio-crude mainly uses H₂ as the reactant and is thus called hydrodeoxygenation (HDO). Typically, the HDO reaction takes place in a fixed-bed reactor at temperatures ranging from 300-400° C. and very high pressure ranging from 30-130 bar (about 30-128 atm), using catalysts consisting of alumina impregnated with cobalt-molybdenum or nickel-molybdenum. The molybdenum center can stabilize a non-ordinary unsaturated site. Oxygen-containing substrates can bind to this site and undergo a series of reactions that result in C—O, C═O, and C═C hydrogenation. In this process, hydrogen is used as a reductant, and oxygen atoms in the feedstock oil can be effectively converted into water (H₂O) in the reactor. Water then needs to be separated from liquid hydrocarbon species to obtain upgraded biofuels.

However, hydrogen is not naturally available. Hydrogen is typically produced by a steam methane reforming (SMR) process. This process normally requires temperatures over 800° C., and pressures ranging from 15-30 bar, resulting in high capital investment and operation costs plus high energy and water consumption (making the process economically unfavorable). In addition, an average of 7 kg CO₂/kg H₂ is generated in the SMR process, leading to more greenhouse gas emissions and potentially adverse environmental impacts. Accordingly, an alternative pathway for deoxygenation of hydrocarbon feedstocks such as crude oil, which can overcome the abovementioned drawbacks, is of great value and urgency.

### BRIEF SUMMARY

In accordance with embodiments described herein, a method for deoxygenation of an oxygen-containing hydrocarbon comprises introducing a methane-containing gas into a reactor, introducing an oxygen-containing hydrocarbon feedstock into the reactor, and reacting the oxygen-containing hydrocarbon feedstock within the reactor in the presence of the methane-containing gas and a catalyst structure to form a hydrocarbon product having an oxygen content that is less than an oxygen content of the oxygen-containing hydrocarbon feedstock.

The catalyst structure can comprise porous support and one or more metals loaded within the porous support, where the porous support comprises one or more of an aluminum oxide material, an aluminosilicate material, an anatase material, a rutile material, and a titanium silicalite material, and further where the one or more metals comprises one or more of Ir, Ga, and Ce.

In further embodiments described herein, a catalyst structure is provided that comprises porous support and one or more metals loaded within the porous support. The porous support can comprise one or more of an aluminum oxide material, an aluminosilicate material, an anatase material, a rutile material, and a titanium silicalite material, and one or more metals can comprise one or more of Ir, Ga, and Ce.

In still further embodiments, a method of forming a catalyst structure comprises dissolving two or more metal salts in water to form a metal precursor solution, loading the metal precursor solution into porous support, drying the porous support loaded with the metal precursor for at least 2 hours at a temperature from about 80° C. to about 120° C., and calcining the dried porous support loaded with metal precursor at a temperature ranging from about 450° C. to about 700° C. and at a heating rate ranging from about 0.5° C./min to about 20° C./min.

The above and still further features and advantages of the present invention will become apparent upon consideration of the following detailed description of specific embodiments thereof.

### DETAILED DESCRIPTION

In the following detailed description, while aspects of the disclosure are disclosed, alternate embodiments of the present disclosure and their equivalents may be devised without parting from the spirit or scope of the present disclosure. It should be noted that any discussion herein regarding “one embodiment”, “an embodiment”, “an exemplary embodiment”, and the like indicate that the embodiment described may include a particular feature, structure, or characteristic and that such particular feature, structure, or characteristic may not necessarily be included in every embodiment. In addition, references to the foregoing do not necessarily comprise a reference to the same embodiment. Finally, irrespective of whether it is explicitly described, one of ordinary skill in the art would readily appreciate that each of the particular features, structures, or characteristics of the given embodiments may be utilized in connection or combination with those of any other embodiment discussed herein.

Various operations may be described as multiple discrete actions or operations in turn, in a manner that is most helpful in understanding the claimed subject matter. However, the order of description should not be construed to imply that these operations are necessarily order-dependent. In particular, these operations may not be performed in the order of presentation. The operations described may be performed in a different order than the described embodiment. Various additional operations may be performed and/or described operations may be omitted in additional embodiments.

For the purposes of the present disclosure, the phrase “A and/or B” means (A), (B), or (A and B). For the purposes of the present disclosure, the phrase “A, B, and/or C” means (A), (B), (C), (A and B), (A and C), (B and C), or (A, B and C).

The terms “comprising,” “including,” “having,” and the like, as used with respect to embodiments of the present disclosure, are synonymous.

The present invention relates to a method of deoxygenation of an oxygen-containing hydrocarbon feedstock. In particular, the present invention relates to a method for light oil quality enhancement and renewable bio-fuel processing. Methods, as described herein, include the formulation of a heterogeneous catalyst and a process of utilizing the catalyst for bio-crude deoxygenation under a methane or natural gas environment for decreasing the oxygen content of such oil feedstock.

The catalyst structure as described herein can comprise porous support including an aluminum oxide (i.e., Al₂O₃), an aluminosilicate material (e.g. zeolite), an anatase material (i.e. TiO₂-anatase), a rutile material (i.e. TiO₂-rutile), and a titanium silicalite (i.e. TS-1) and one or more metals loaded in the porous support structure. One or more metals can comprise Ir (iridium), Ga (gallium), Ce (cerium), and any two or more combinations thereof. Each metal loaded in the porous support structure is present in an amount from about 0.1 wt % to about 10 wt %. It is noted that the term weight percentage (wt %) of a metal as applied to being within a catalyst structure, as described herein, refers to the weight of a particular metal element divided by the total weight of the catalyst support and then multiplied by 100 (to obtain a percentage value).

Further, in the following embodiments, a process of forming the catalyst structure is provided.

Further, in the following embodiments, a method for the deoxygenation of an oxygen-containing hydrocarbon feedstock (e.g., a bio-crude liquid) in the presence of the catalyst structure and specific gas environment is described herein, which can effectively lower the oxygen content of the feedstock with simple process operation.

The implementation of the embodiments can further result in advantageous properties of products including but not limited to: a decrease in oxygen content, decrease in coke content and decrease in TAN (total acid number).

The hydrocarbon feedstock can comprise any hydrocarbon that contains oxygen, such as a heavy oil feedstock, a medium oil feedstock, or a light oil feedstock. A particularly suitable form of the methods as described herein is the deoxygenation of a bio-crude liquid. A bio-crude oil or bio-crude fuel, as described herein, is a liquified form or liquid product formed from flash pyrolysis of biomass. For example, bio-crude can be formed from pyrolysis of some form of solid or semi-solid organic biomass material including, without limitation, solid bio-waste such as municipal waste (e.g., municipal solid waste or MSW) including organic material, lignin-based sources including agricultural and/or forestry residues such as corn stover, lignin feedstocks from wood (e.g., wood pellets, wood mill residues, etc.) and/or any other suitable sources of lignin, algae, industrial waste streams including organic material, etc., as well as any other suitable types of biomass material. Bio-crude is a complex mixture of oxygenated organic compounds with a wide molecular weight range. The oxygen content in bio-crude oils ranges from about 2 wt % to about 20 wt %, while the density ranges from 0.9 to 1.2 g/mL. In addition, bio-crude oils are usually viscous, and their viscosity can be 10 to 10,000 times higher than that of typical diesel. Based on the deoxygenation performances of this process, a bio-crude oil having an oxygen content of at least about 2 wt % (e.g., from about 2 wt % to about 20 wt %, such as around 2.26 wt %) is converted to a product with a trace amount of oxygen. For example, the converted product can have an amount of oxygen of less than about 2 wt %, or no greater than about 1 wt %, or no greater than about 0.8 wt %, or no greater than about 0.7 wt %, or even no greater than about 0.5 wt %. It is noted that the term weight percentage (wt %) of oxygen (or any other component) as applied to a hydrocarbon product (e.g., oil product, such as a converted or final oil product from a crude oil feedstock), as described herein, refers to the mass of the component divided by the total mass of the hydrocarbon product and then multiplied by 100 (to obtain a percentage value).

In accordance with example embodiments, unique catalyst structures are described herein for use in the combination with processes for deoxygenation of a bio-crude or other oxygen-containing hydrocarbon feedstock utilizing methane or natural gas resources and one or more of such catalyst structures to achieve a product with lower oxygen content.

The use of some methane, rather than hydrogen, in the oil deoxygenation process obviates the need for an economically unfavorable hydrotreating step at high pressures and temperatures. Methane is the main component in natural gas, which is a naturally occurring resource with underestimated values. The production of natural gas has also increased significantly in the past decades while the price is decreasing continuously. The use of methane or natural gas as a hydrogen donor for oil deoxygenation to produce high-value low oxygen content products provides a more environmentally and economically friendly process. In addition, this also provides the added value of natural gas, which is highly beneficial and profitable for the petroleum and natural gas industry. It has been observed that methane can be effectively activated, which facilitates the deoxygenation process. This method provides a transformational way for oil deoxygenation in the biofuel industry.

In addition, the catalyst structures described herein facilitate the oil deoxygenation process in the presence of methane or natural gas at low temperatures (e.g., in the range of 350-450° C., such as about 400° C.) and pressures (e.g., in the range of 1-50 atm, such as about 30 atm) in the presence of such catalyst structures.

### Catalyst Structures

In accordance with the present invention, a catalyst structure for use in the deoxygenation of a bio-crude or other hydrocarbon feedstock is provided that comprises one or the combination of mono or multi-metallic (e.g., bimetallic) active components loaded on highly porous supports for oil deoxygenation in a methane environment. It is noted that the catalyst structures described herein can also be used for oxygen removal in other gaseous environments such as H₂ and N₂ environments, although utilizing a methane environment is preferred.

The catalyst structure can be synthesized by impregnating or doping a suitable support material with two or more metals. Suitably porous support material can be an aluminum oxide (i.e., Al₂O₃), an aluminosilicate material (e.g. zeolite), an anatase material (i.e. TiO₂-anatase), a rutile material (i.e. TiO₂-rutile), and a titanium silicalite material (i.e. TS-1). Suitable metals that can be loaded on the porous support material by impregnation or doping include any one or more (and preferably any two or more) from the following group: Iridium (Ir), gallium (Ga), and cerium (Cc). Each metal dopant or the combination of metal dopants can be provided within the catalyst structure in an amount ranging from 0.1-10 wt % (i.e., based upon the total weight of the catalyst structure). Specific examples are provided herein of different metal loadings for catalyst structures.

The porous support material can be doped with a suitable amount of one or more metals in the following manner. One or more metal salts can be dissolved in deionized water to form an aqueous solution at a suitable concentration(s) within the solution. Metal precursor salts that can be used to form the catalyst structure include, without limitation, chlorides and nitrates. The one or more metal precursors in the solution are then loaded into the porous support material to achieve a desired amount of metals within the catalyst structure (e.g., from 0.1-10 wt %). Any suitable loading process can be performed to load metals within the porous support material. Some non-limiting examples of metal loading processes include IWI (incipient wetness impregnation, where an active metal precursor is first dissolved in an aqueous or organic solution, the metal-containing solution is then added to catalyst support containing the same pore volume as the added solution volume, where capillary action draws the solution into the pores); WI (wet impregnation, where more liquid than the IWI volume is added to the support, and the solvent is then removed by evaporation); CA assisted WI (wet impregnation with the assist of citric acid, where more liquid than the IWI volume is added to the support, and the solvent is then removed by evaporation, citric acid is removed by the following calcination); and DPU (deposition precipitation using urea).

Depending upon the particular loading process, the resultant metal-loaded catalyst structure can be dried at a temperature from about 80° C. to about 120° C. for some time from about 2 hours to about 24 hours. The dried catalyst structure can then be calcined under air at a temperature ranging from 450-700° C. and at a suitable ramped or stepwise increased heating rate (e.g., a heating rate of about 0.5-20° C./min), where such calcination temperatures, times, and heating rates can be modified depending upon the type or types of metals doped into the catalyst structure as well as reaction conditions associated with the use of the catalyst structure.

The resultant metal-doped catalyst structure is suitable for use in oil deoxygenation under a methane environment in processes as described herein. The catalyst structure can be processed into a granular form with a granule size desired for a particular operation. The catalyst structure can also be formed into any other suitable configuration. For example, the catalyst structure in a powder form or a pelleted form can be utilized in a batch reactor system or a continuous flow reactor system. When utilizing a continuous reactor (i.e., continuous flow reactor), the liquid hourly space velocity (LHSV) of the hydrocarbon feedstock within the reactor can be within a range from about 0.1 h⁻¹ to about 10 h⁻¹.

### Systems and Methods for Oil Deoxygenation Utilizing the Catalyst Structures

The conversion of oxygen-containing hydrocarbon species toward hydrocarbon products having smaller (e.g., trace) amounts of oxygent in the products can be fine-tuned using catalyst structures as described herein and under a methane environment.

Methane, as a main component in natural gas, is particularly useful for oil deoxygenation in the presence of catalysts described herein. Methane is typically regarded as chemically inert due to its stable structure, and methane activation has been a challenge in natural gas utilization. However, it has been determined that methane utilization can be significantly enhanced with the assistance of the catalysts as described herein.

Some examples of catalyst structures and processes used to deoxygenate a crude-oil feedstock are now described. In each example, the crude-oil feedstock has an oxygen content of at least about 2 wt % (i.e., based upon total weight of the feedstock), and the feedstock is converted to a hydrocarbon (oil) product containing a reduced amount of oxygen (i.e., less than about 2 wt % and, in particular, no greater than about 1 wt %, or no greater than about 0.8 wt %, or even no greater than about 0.7 wt %, or even no greater than about 0.5 wt %).

### Example 1

1 wt % Ir-1 wt % Ga-5 wt % Ce/TiO₂-anatase catalyst was prepared in the following manner. A homemade TiO₂, anatase support structure in powder form was used without further treatment. The metal salts include x moles of gallium (III) nitrate hydrate (Ga(NO₃)₃·xH₂O) and y moles of cerium (III) nitrate hexahydrate (Ce(NO₃)₃·6H₂O), as well as 1.5(x+y) moles of citric acid powder, was dissolved in deionized water to form a precursor solution. The TiO₂ support was then mixed with the precursor solution, followed by stirring at 80° C. until most of the water was evaporated. The obtained wet powder was first dried in an oven at 110° C. overnight, followed by calcination at 450° C. with the temperature increased at a rate of 0.5° C./min in a muffle furnace under an air atmosphere for 4 hours. The calcined powder (Ga—Ce/TiO₂-anatase) was then added to an aqueous solution containing IrCl₃ and urea at the following ratio: 1.06 g of Ga—Ce/TiO₂, 1.04 10⁻³ M IrCl₃, 1.04 M urea, and 50 mL water. The resultant suspension was stirred at 80° C. for 16 hours to achieve Ir loading. The resultant powder was separated by a centrifuge and washed with deionized water, and the operation was repeated 4 times. The obtained wet powder was first dried in an oven at 110° C. overnight, followed by calcination at 450° C. with the temperature climbing at a rate of 10° C./min in a muffle furnace under an air atmosphere for 4 hours. The catalyst is denoted as 1% Ir-1% Ga-5% Ce/TiO₂-A.

Bio-crude was pumped into a fixed bed reactor at a rate of 0.09 mL/min. 5 mL Ir—Ga—Ce/TiO₂-anatase catalyst was loaded onto the reactor. The reaction was carried out under 400° C. and a gas atmosphere of 2.7 MPa CH₄ and 0.3 MPa N₂. The gas feeding rate was 100 sccm and the reaction lasted 3 hours. The outlet gas was directly connected to a micro-GC instrument for further analysis. The liquid product was collected in a stainless steel container cooled at −4° C. by a chiller. The solid product was collected together with the spent catalyst after the reaction, and quantified by TGA analysis. Methane conversion, gas yield, liquid yield, coke yield, and overall mass balance were measured and listed in Table 1. The composition of gas products is listed in Table 2, and the total acid number (TAN) and oxygen content data are listed in Table 3.

### Example 2

1 wt % Ir-1 wt % Ga/TiO₂-anatase catalyst was prepared in the following manner. A homemade TiO₂, anatase support structure in powder form was used without further treatment. The metal salts x moles of gallium (III) nitrate hydrate (Ga(NO₃)₃·xH₂O) and 1.5× moles of citric acid powder, were dissolved in deionized water to form a precursor solution. The TiO₂ support was then mixed with the precursor solution, followed by stirring at 80° C. until most of the water was evaporated. The obtained wet powder was first dried in an oven at 110° C. overnight, followed by calcination at 450° C. with the temperature climbing at a rate of 0.5° C./min in a muffle furnace under an air atmosphere for 4 hours. The calcined powder (Ga/TiO₂) was then added to an aqueous solution containing IrCl₃ and urea at the following ratio: 1.01 g of Ga/TiO₂, 1.04 10⁻³ M IrCl₃, 1.04 M urea, and 50 mL water. The resultant suspension was stirred at 80° C. for 16 hours to achieve Ir loading. The resultant powder was separated by a centrifuge and washed with deionized water, the operation was repeated 4 times. The obtained wet powder was first dried in an oven at 110° C. overnight, followed by calcination at 450° C. with the temperature climbing at a rate of 10° C./min in a muffle furnace under an air atmosphere for 4 hours. The catalyst is denoted as 1% Ir-1% Ga/TiO₂-A.

Bio-crude was pumped into a fixed bed reactor at a rate of 0.09 mL/min. 5 mL Ir—Ga/TiO₂-anatase catalyst was loaded onto the reactor. The reaction was carried out under 400° C. and a gas atmosphere of 2.7 MPa CH₄ and 0.3 MPa N₂. The gas feeding rate was 100 sccm and the reaction lasted 3 hours. The outlet gas was directly connected to a micro-GC instrument for further analysis. The liquid product was collected in a stainless steel container cooled at −4° C. by a chiller. The solid product was collected together with the spent catalyst after the reaction, and quantified by TGA analysis. Methane conversion, gas yield, liquid yield, coke yield, and overall mass balance were measured and listed in Table 1. The composition of gas products is listed in Table 2, and the total acid number (TAN) and oxygen content data are listed in Table 3.

### Example 3

1 wt % Ir-5 wt % Ce/TiO₂-anatase catalyst was prepared in the following manner. A homemade TiO₂, anatase support structure in powder form was used without further treatment. The metal salts y moles of cerium (III) nitrate hexahydrate (Ce(NO₃)₃·6H₂O), as well as 1.5y moles of citric acid powder, was dissolved in deionized water to form a precursor solution. The TiO₂ support was then mixed with the precursor solution, followed by stirring at 80° C. until most of the water was evaporated. The obtained wet powder was first dried in an oven at 110° C. overnight, followed by calcination at 450° C. with the temperature climbing at a rate of 0.5° C./min in a muffle furnace under an air atmosphere for 4 hours. The calcined powder (Ga—Ce/TiO₂-anatase) was then added to an aqueous solution containing IrCl₃ and urea at the following ratio: 1.05 g of Ce/TiO₂, 1.04 10⁻³ M IrCl₃, 1.04 M urea, and 50 mL water. The resultant suspension was stirred at 80° C. for 16 hours to achieve Ir loading. The resultant powder was separated by a centrifuge and washed with deionized water, the operation was repeated 4 times. The obtained wet powder was first dried in an oven at 110° C. overnight, followed by calcination at 450° C. with the temperature climbing at a rate of 10° C./min in a muffle furnace under an air atmosphere for 4 hours. The catalyst is denoted as 1% Ir-5% Ce/TiO₂-A.

Bio-crude was pumped into a fixed bed reactor at a rate of 0.09 mL/min. 5 mL Ir—Ce/TiO₂-anatase catalyst was loaded onto the reactor. The reaction was carried out under 400° C. and a gas atmosphere of 2.7 MPa CH₄ and 0.3 MPa N₂. The gas feeding rate was 100 sccm and the reaction lasted 3 hours. The outlet gas was directly connected to a micro-GC instrument for further analysis. The liquid product was collected in a stainless steel container cooled at −4° C. by a chiller. The solid product was collected together with the spent catalyst after the reaction, and quantified by TGA analysis. Methane conversion, gas yield, liquid yield, coke yield, and overall mass balance were measured and listed in Table 1. The composition of gas products is listed in Table 2, and the total acid number (TAN) and oxygen content data are listed in Table 3.

### Example 4

1 wt % Ga-5 wt % Ce/TiO₂-anatase catalyst was prepared in the following manner. A homemade TiO₂, anatase support structure in powder form was used without further treatment. The metal salts x moles of gallium (III) nitrate hydrate (Ga(NO₃)₃·xH₂O) and y moles of cerium (III) nitrate hexahydrate (Ce(NO₃)₃·6H₂O), as well as 1.5(x+y) moles of citric acid powder, was dissolved in deionized water to form a precursor solution. The TiO₂ support was then mixed with the precursor solution, followed by stirring at 80° C. until most of the water was evaporated. The obtained wet powder was first dried in an oven at 110° C. overnight, followed by calcination at 450° C. with the temperature climbing at a rate of 0.5° C./min in a muffle furnace under an air atmosphere for 4 hours. The catalyst is denoted as 1% Ga-5% Ce/TiO₂-A.

Bio-crude was pumped into a fixed bed reactor at a rate of 0.09 mL/min. 5 mL Ga—Ce/TiO₂-anatase catalyst was loaded onto the reactor. The reaction was carried out under 400° C. and a gas atmosphere of 2.7 MPa CH₄ and 0.3 MPa N₂. The gas feeding rate was 100 sccm and the reaction lasted 3 hours. The outlet gas was directly connected to a micro-GC instrument for further analysis. The liquid product was collected in a stainless steel container cooled at −4° C. by a chiller. The solid product was collected together with the spent catalyst after the reaction, and quantified by TGA analysis. Methane conversion, gas yield, liquid yield, coke yield, and overall mass balance were measured and listed in Table 1. The composition of gas products is listed in Table 2, and the total acid number (TAN) and oxygen content data are listed in Table 3.

### Example 5

1 wt % Ir-1 wt % Ga-5 wt % Ce/TiO₂-rutile catalyst was prepared in the following manner. A TiO₂ (Titanium (IV) oxide, rutile, powder, <5 μm) support structure in powder form obtained from Sigma-Aldrich was used without further treatment. The metal salts x moles of gallium (III) nitrate hydrate (Ga(NO₃)₃·xH₂O) and y moles of cerium (III) nitrate hexahydrate (Ce(NO₃)₃-6H₂O), as well as 1.5(x+y) moles of citric acid powder, was dissolved in deionized water to form a precursor solution. The TiO₂ support was then mixed with the precursor solution, followed by stirring at 80° C. until most of the water was evaporated. The obtained wet powder was first dried in an oven at 110° C. overnight, followed by calcination at 450° C. with the temperature climbing at a rate of 0.5° C./min in a muffle furnace under an air atmosphere for 4 hours. The calcined powder (Ga—Ce/TiO₂-rutile) was then added to an aqueous solution containing IrCl₃ and urea at the following ratio: 1.06 g of Ga—Ce/TiO₂-rutile, 1.04 10-3 M IrCl₃, 1.04 M urea, and 50 mL water. The resultant suspension was stirred at 80° C. for 16 hours to achieve Ir loading. The resultant powder was separated by a centrifuge and washed with deionized water, the operation was repeated 4 times. The obtained wet powder was first dried in an oven at 110° C. overnight, followed by calcination at 450° C. with the temperature climbing at a rate of 10° C./min in a muffle furnace under an air atmosphere for 4 hours. The catalyst is denoted as 1% Ir-1% Ga-5% Ce/TiO₂—R.

Bio-crude was pumped into a fixed bed reactor at a rate of 0.09 mL/min. 5 mL Ir—Ga—Ce/TiO₂-rutile catalyst was loaded onto the reactor. The reaction was carried out under 400° C. and a gas atmosphere of 2.7 MPa CH₄ and 0.3 MPa N₂. The gas feeding rate was 100 sccm and the reaction lasted 3 hours. The outlet gas was directly connected to a micro-GC instrument for further analysis. The liquid product was collected in a stainless steel container cooled at −4° C. by a chiller. The solid product was collected together with the spent catalyst after the reaction, and quantified by TGA analysis. Methane conversion, gas yield, liquid yield, coke yield, and overall mass balance were measured and listed in Table 1. The composition of gas products is listed in Table 2, and the total acid number (TAN) and oxygen content data are listed in Table 3.

### Example 6

1 wt % Ir-1 wt % Ga-5 wt % Ce/TS-1 catalyst was prepared in the following manner. A TS-1 (Titanium silicalite molecular sieve, SiO2/TiO₂ ratio≥25, 0.3-0.5 μm) support structure in powder form obtained from Sigma-Aldrich was used without further treatment. The metal salts x moles of gallium (III) nitrate hydrate (Ga(NO₃)₃·xH₂O) and y moles of cerium (III) nitrate hexahydrate (Ce(NO₃)₃·6H₂O), as well as 1.5(x+y) moles of citric acid powder, was dissolved in deionized water to form a precursor solution. The TS-1 support was then mixed with the precursor solution, followed by stirring at 80° C. until most of the water was evaporated. The obtained wet powder was first dried in an oven at 110° C. overnight, followed by calcination at 450° C. with the temperature climbing at a rate of 0.5° C./min in a muffle furnace under an air atmosphere for 4 hours. The calcined powder (Ga—Ce/TS-1) was then added to an aqueous solution containing IrCl₃ and urea at the following ratio: 1.06 g of Ga—Ce/TS-1, 1.04 103 M IrCl₃, 1.04 M urea, and 50 mL water. The resultant suspension was stirred at 80° C. for 16 hours to achieve Ir loading. The resultant powder was separated by a centrifuge and washed with deionized water, the operation was repeated 4 times. The obtained wet powder was first dried in an oven at 110° C. overnight, followed by calcination at 450° C. with the temperature climbing at a rate of 10° C./min in a muffle furnace under an air atmosphere for 4 hours. The catalyst is denoted as 1% Ir-1% Ga-5% Ce/TS-1.

Bio-crude was pumped into a fixed bed reactor at a rate of 0.09 mL/min. 5 mL Ir—Ga—Ce/TS-1 catalyst was loaded onto the reactor. The reaction was carried out under 400° C. and a gas atmosphere of 2.7 MPa CH₄ and 0.3 MPa N₂. The gas feeding rate was 100 sccm and the reaction lasted 3 hours. The outlet gas was directly connected to a micro-GC instrument for further analysis. The liquid product was collected in a stainless steel container cooled at −4° C. by a chiller. The solid product was collected together with the spent catalyst after the reaction, and quantified by TGA analysis. Methane conversion, gas yield, liquid yield, coke yield, and overall mass balance were measured and listed in Table 1. The composition of gas products is listed in Table 2, and the total acid number (TAN) and oxygen content data are listed in Table 3.

### Example 7

1 wt % Ga-5 wt % Ce/Al₂O₃ catalyst was prepared in the following manner. An Al₂O₃ (Aluminum oxide, high surface area, ⅛″ pellets) support structure in pellet form obtained from Sigma-Aldrich was used without further treatment. The metal salts gallium (III) nitrate hydrate (Ga(NO₃)₃·xH₂O) and cerium (III) nitrate hexahydrate (Ce(NO₃)₃·6H₂O) was dissolved in deionized water to form a precursor solution. The Al₂O₃ support was then mixed with the precursor solution, followed by wet impregnation (WI) to achieve metal loading. The resultant pellet was firstly dried in an oven overnight at 80° C., then calcined at 550° C. with the temperature climbing at a rate of 10° C./min in a muffle furnace under an air atmosphere for 4 hours. The catalyst is denoted as 1% Ga-5% Ce/Al₂O₃.

Bio-crude was pumped into a fixed bed reactor at a rate of 0.09 mL/min. 5 mL Ga—Ce/Al₂O₃ catalyst was loaded onto the reactor. The reaction was carried out under 400° C. and a gas atmosphere of 2.7 MPa CH₄ and 0.3 MPa N₂. The gas feeding rate was 100 sccm and the reaction lasted 3 hours. The outlet gas was directly connected to a micro-GC instrument for further analysis. The liquid product was collected in a stainless steel container cooled at −4° C. by a chiller. The solid product was collected together with the spent catalyst after the reaction, and quantified by TGA analysis. Methane conversion, gas yield, liquid yield, coke yield, and overall mass balance were measured and listed in Table 1. The composition of gas products is listed in Table 2, and the total acid number (TAN) and oxygen content data are listed in Table 3.

### Example 8

1 wt % Ga-5 wt % Ce/UZSM-5 catalyst was prepared in the following manner. A homemade UZSM-5 zeolite support structure in pellet form was used without further treatment. The metal salts gallium (III) nitrate hydrate (Ga(NO₃)₃·xH₂O) and cerium (III) nitrate hexahydrate (Cc (NO₃) 3.6H₂O) was dissolved in deionized water to form a precursor solution. The UZSM-5 support was then mixed with the precursor solution, followed by wet impregnation (WI) to achieve metal loading. The resultant pellet was firstly dried in an oven overnight at 80° C., then calcined at 550° C. with the temperature climbing at a rate of 10° C./min in a muffle furnace under an air atmosphere for 4 hours. The catalyst is denoted as 1% Ga-5% Ce/UZSM-5.

Bio-crude was pumped into a fixed bed reactor at a rate of 0.09 mL/min. 5 mL Ga—Ce/UZSM-5 catalyst was loaded onto the reactor. The reaction was carried out under 400° C. and a gas atmosphere of 2.7 MPa CH₄ and 0.3 MPa N₂. The gas feeding rate was 100 sccm and the reaction lasted 3 hours. The outlet gas was directly connected to a micro-GC instrument for further analysis. The liquid product was collected in a stainless steel container cooled at −4° C. by a chiller. The solid product was collected together with the spent catalyst after the reaction, and quantified by TGA analysis. Methane conversion, gas yield, liquid yield, coke yield, and overall mass balance were measured and listed in Table 1. The composition of gas products is listed in Table 2, and the total acid number (TAN) and oxygen content data are listed in Table 3.

### Example 9

1 wt % Ir-1 wt % Ga-10 wt % Ce/TiO₂-anatase catalyst was prepared in the following manner. A homemade TiO₂, anatase support structure in powder form was used without further treatment. The metal salts x moles of gallium (III) nitrate hydrate (Ga(NO₃)₃·xH₂O) and y moles of cerium (III) nitrate hexahydrate (Ce(NO₃)₃·6H₂O), as well as 1.5(x+y) moles of citric acid powder, was dissolved in deionized water to form a precursor solution. The TiO₂ support was then mixed with the precursor solution, followed by stirring at 80° C. until most of the water was evaporated. The obtained wet powder was first dried in an oven at 120° C. for 2 hours, followed by calcination at 450° C. with the temperature climbing at a rate of 0.5° C./min in a muffle furnace under an air atmosphere for 4 hours. The calcined powder (Ga—Ce/TiO₂-anatase) was then added to an aqueous solution containing IrCl₃ and urea at the following ratio: 1.11 g of Ga—Ce/TiO₂, 1.04 10⁻³ M IrCl₃, 1.04 M urea, and 50 mL water. The resultant suspension was stirred at 80° C. for 16 hours to achieve Ir loading. The resultant powder was separated by a centrifuge and washed with deionized water, the operation was repeated 4 times. The obtained wet powder was first dried in an oven at 120° C. for 2 hours, followed by calcination at 450° C. with the temperature climbing at a rate of 20° C./min in a muffle furnace under an air atmosphere for 4 hours. The catalyst is denoted as 1% Ir-1% Ga-10% Ce/TiO₂-A.

Bio-crude was pumped into a fixed bed reactor at a rate of 0.01 mL/min. 6 mL Ir—Ga—Ce/TiO₂-anatase catalyst was loaded onto the reactor. The reaction was carried out under 450° C. and a gas atmosphere of 0.09 MPa CH₄ and 0.01 MPa N₂. The gas feeding rate was 100 sccm and the reaction lasted 3 hours. The outlet gas was directly connected to a micro-GC instrument for further analysis. The liquid product was collected in a stainless steel container cooled at −4° C. by a chiller. The solid product was collected together with the spent catalyst after the reaction, and quantified by TGA analysis. Methane conversion, gas yield, liquid yield, coke yield, and overall mass balance were measured and listed in Table 1. The composition of gas products is listed in Table 2, and the total acid number (TAN) and oxygen content data are listed in Table 3.

### Example 10

0.1 wt % Ir-1 wt % Ga-5 wt % Ce/TiO₂-anatase catalyst was prepared in the following manner. A homemade TiO₂, anatase support structure in powder form was used without further treatment. The metal salts x moles of gallium (III) nitrate hydrate (Ga(NO₃)₃·xH₂O) and y moles of cerium (III) nitrate hexahydrate (Ce(NO₃)₃·6H₂O), as well as 1.5(x+y) moles of citric acid powder, was dissolved in deionized water to form a precursor solution. The TiO₂ support was then mixed with the precursor solution, followed by stirring at 80° C. until most of the water was evaporated. The obtained wet powder was first dried in an oven at 120° C. for 2 hours, followed by calcination at 700° C. with the temperature climbing at a rate of 0.5° C./min in a muffle furnace under an air atmosphere for 4 hours. The calcined powder (Ga—Ce/TiO₂-anatase) was then added to an aqueous solution containing IrCl₃ and urea at the following ratio: 1.06 g of Ga—Ce/TiO₂, 1.04 10⁻⁴M IrCl₃, 0.104 M urea, and 50 mL water. The resultant suspension was stirred at 80° C. for 16 hours to achieve Ir loading. The resultant powder was separated by a centrifuge and washed with deionized water, the operation was repeated 4 times. The obtained wet powder was first dried in an oven at 120° C. for 2 hours, followed by calcination at 700° C. with the temperature climbing at a rate of 20° C./min in a muffle furnace under an air atmosphere for 4 hours. The catalyst is denoted as 0.1% Ir-1% Ga-5% Ce/TiO₂-A.

Bio-crude was pumped into a fixed bed reactor at a rate of 1 mL/min. 6 mL Ir—Ga—Ce/TiO₂-anatase catalyst was loaded onto the reactor. The reaction was carried out under 350° C. and a gas atmosphere of 4.5 MPa CH₄ and 0.5 MPa N₂. The gas feeding rate was 100 sccm and the reaction lasted 3 hours. The outlet gas was directly connected to a micro-GC instrument for further analysis. The liquid product was collected in a stainless steel container cooled at −4° C. by a chiller. The solid product was collected together with the spent catalyst after the reaction, and quantified by TGA analysis. Methane conversion, gas yield, liquid yield, coke yield, and overall mass balance were measured and listed in Table 1. The composition of gas products is listed in Table 2, and the total acid number (TAN) and oxygen content data are listed in Table 3.

TABLE 1
Overall analysis results in Examples 1-10.

| | | | Gas | Liquid | Coke | Overall |
|---|---|---|---|---|---|---|
| | | | yield | yield | yield | mass |
| Example | Catalyst | Gas | (wt %) | (wt %) | (wt %) | balance (%) |
| 1 | 1% Ir-1% Ga- | 2.7 MPa CH₄, 0.3 | 3.54 | 95.47 | 0.44 | 99.45 |
| | 5% Ce/TiO₂-A | MPa N₂ | | | | |
| 2 | 1% Ir- | 2.7 MPa CH₄, 0.3 | 3.10 | 95.06 | 3.23 | 99.39 |
| | 1% Ga/TiO₂-A | MPa N₂ | | | | |
| 3 | 1% Ir- | 2.7 MPa CH₄, 0.3 | 2.80 | 94.18 | 2.32 | 99.30 |
| | 5% Ce/TiO₂-A | MPa N₂ | | | | |
| 4 | 1% Ga- | 2.7 MPa CH₄, 0.3 | 2.83 | 95.13 | 1.40 | 99.36 |
| | 5% Ce/TiO₂-A | MPa N₂ | | | | |
| 5 | 1% Ir-1% Ga- | 2.7 MPa CH₄, 0.3 | 3.24 | 94.20 | 1.75 | 99.19 |
| | 5% Ce/TiO₂-R | MPa N₂ | | | | |
| 6 | 1% Ir-1% Ga- | 2.7 MPa CH₄, 0.3 | 3.27 | 93.96 | 2.03 | 99.26 |
| | 1% Ce/TS-1 | MPa N₂ | | | | |
| 7 | 1% Ga- | 2.7 MPa CH₄, 0.3 | 2.54 | 93.85 | 2.89 | 99.28 |
| | 5% Ce/Al₂O₃ | MPa N₂ | | | | |
| 8 | 1% Ga- | 2.7 MPa CH₄, 0.3 | 6.03 | 92.04 | 1.16 | 99.23 |
| | 5% Ce/UZSM-5 | MPa N₂ | | | | |
| 9 | 1% Ir-1% Ga- | 4.5 MPa CH₄, | 6.72 | 90.55 | 2.15 | 99.42 |
| | 10% Ce/TiO₂-A | 0.5 MPa N₂ | | | | |
| 10 | 0.1% Ir-1% Ga- | 0.09 MPa CH₄, | 0.56 | 99.06 | 0.15 | 99.77 |
| | 5% Ce/TiO₂-A | 0.01 MPa N₂ | | | | |

TABLE 2
Gas analysis of products in Example 1-10.

| | | | | | | CO₂ |
|---|---|---|---|---|---|---|
| | | | CH₄ | H₂ | C2-C5 | yield |
| Ex- | | | conv. | yield | yield | (wt |
| ample | Catalyst | Gas | (wt %) | (wt %) | (wt %) | %) |
| 1 | 1% Ir-1% Ga- | 2.7 MPa CH₄, | 1.66 | 0.40 | 0.40 | 2.46 |
| | 5% Ce/TiO₂-A | 0.3 MPa N₂ | | | | |
| 2 | 1% Ir- | 2.7 MPa CH₄, | 1.87 | 0.56 | 0.24 | 1.77 |
| | 1% Ga/TiO₂- | 0.3 MPa N₂ | | | | |
| | A | | | | | |
| 3 | 1% Ir- | 2.7 MPa CH₄, | 1.16 | 0.39 | 0.25 | 1.87 |
| | 5% Ce/TiO₂-A | 0.3 MPa N₂ | | | | |
| 4 | 1% Ga- | 2.7 MPa CH₄, | 0.20 | 0.03 | BDL | 2.52 |
| | 5% Ce/TiO₂-A | 0.3 MPa N₂ | | | | |
| 5 | 1% Ir-1% Ga- | 2.7 MPa CH₄, | 1.71 | 0.34 | 0.35 | 2.54 |
| | 5% Ce/TiO₂-R | 0.3 MPa N₂ | | | | |
| 6 | 1% Ir-1% | 2.7 MPa CH₄, | 1.45 | 0.21 | 0.63 | 2.21 |
| | Ga-1% | 0.3 MPa N₂ | | | | |
| | Ce/TS-1 | | | | | |
| 7 | 1% Ga- | 2.7 MPa CH₄, | 0.30 | 0.05 | 0.36 | 2.13 |
| | 5% Ce/Al₂O₃ | 0.3 MPa N₂ | | | | |
| 8 | 1% Ga- | 2.7 MPa CH₄, | 0.45 | 0.03 | 5.08 | 0.92 |
| | 5% Ce/UZSM- | 0.3 MPa N₂ | | | | |
| | 5 | | | | | |
| 9 | 1% Ir-1% Ga- | 4.5 MPa CH₄, | 2.75 | 0.63 | 3.23 | 2.86 |
| | 10% Ce/TiO₂- | 0.5 MPa N₂ | | | | |
| | A | | | | | |
| 10 | 0.1% Ir- | 0.09 MPa CH₄, | 0.24 | BDL | 0.35 | 0.21 |
| | 1% Ga- | 0.01 MPa N₂ | | | | |
| | 5% Ce/TiO₂-A | | | | | |

TABLE 3
Oxygen content & TAN data in Example 1-10.

| | | | Oxygen | TAN |
|---|---|---|---|---|
| | | | content | (mg KOH/ |
| Example | Catalyst | Gas | (wt %) | g sample) |
| Feedstock | / | / | 2.26 | 34.22 |
| 1 | 1% Ir-1% Ga- | 2.7 MPa CH₄, | Trace | 0.20 |
| | 5% Ce/TiO₂-A | 0.3 MPa N₂ | | |
| 2 | 1% Ir- | 2.7 MPa CH₄, | 0.65 | 2.55 |
| | 1% Ga/TiO₂-A | 0.3 MPa N₂ | | |
| 3 | 1% Ir- | 2.7 MPa CH₄, | 0.36 | 0.64 |
| | 5% Ce/TiO₂-A | 0.3 MPa N₂ | | |
| 4 | 1% Ga- | 2.7 MPa CH₄, | Trace | 0.33 |
| | 5% Ce/TiO₂-A | 0.3 MPa N₂ | | |
| 5 | 1% Ir-1% Ga- | 2.7 MPa CH₄, | Trace | 0.23 |
| | 5% Ce/TiO₂-R | 0.3 MPa N₂ | | |
| 6 | 1% Ir-1% Ga- | 2.7 MPa CH₄, | Trace | 0.27 |
| | 1% Ce/TS-1 | 0.3 MPa N₂ | | |
| 7 | 1% Ga- | 2.7 MPa CH₄, | 0.35 | 0.55 |
| | 5% Ce/Al₂O₃ | 0.3 MPa N₂ | | |
| 8 | 1% Ga- | 2.7 MPa CH₄, | 0.53 | 1.09 |
| | 5% Ce/UZSM-5 | 0.3 MPa N₂ | | |
| 9 | 1% Ir-1% Ga- | 4.5 MPa CH₄, | Trace | 0.09 |
| | 10% Ce/TiO₂-A | 0.5 MPa N₂ | | |
| 10 | 0.1% Ir-1% Ga- | 0.09 MPa CH₄, | 1.91 | 26.37 |
| | 5% Ce/TiO₂-A | 0.01 MPa N₂ | | |

### Example 11

1 wt % Ir-1 wt % Ga-5 wt % Ce/TiO₂-anatase catalyst was prepared in the following manner. A homemade TiO₂, anatase support structure in powder form was used without further treatment. The metal salts x moles of gallium (III) nitrate hydrate (Ga(NO₃)₃·xH₂O) and y moles of cerium (III) nitrate hexahydrate (Ce(NO₃)₃·6H₂O), as well as 1.5(x+y) moles of citric acid powder, was dissolved in deionized water to form a precursor solution. The TiO₂ support was then mixed with the precursor solution, followed by stirring at 80° C. until most of the water was evaporated. The obtained wet powder was first dried in an oven at 110° C. overnight, followed by calcination at 450° C. with the temperature climbing at a rate of 0.5° C./min in a muffle furnace under an air atmosphere for 4 hours. The calcined powder (Ga—Ce/TiO₂-anatase) was then added to an aqueous solution containing IrCl₃ and urea at the following ratio: 1.06 g of Ga—Ce/TiO₂, 1.04 10⁻³ M IrCl₃, 1.04 M urea, and 50 mL water. The resultant suspension was stirred at 80° C. for 16 hours to achieve Ir loading. The resultant powder was separated by a centrifuge and washed with deionized water, the operation was repeated 4 times. The obtained wet powder was first dried in an oven at 110° C. overnight, followed by calcination at 450° C. with the temperature climbing at a rate of 10° C./min in a muffle furnace under an air atmosphere for 4 hours. The catalyst is denoted as 1% Ir-1% Ga-5% Ce/TiO₂-A.

Bio-crude was pumped into a fixed bed reactor at a rate of 0.09 mL/min. 5 mL Ir—Ga—Ce/TiO₂-anatase catalyst was loaded onto the reactor. The reaction was carried out under 400° C. and a gas atmosphere of 3.0 MPa N₂. The gas feeding rate was 100 sccm and the reaction lasted 3 hours. The outlet gas was directly connected to a micro-GC instrument for further analysis. The liquid product was collected in a stainless steel container cooled at −4° C. by a chiller. The solid product was collected together with the spent catalyst after the reaction, and quantified by TGA analysis. Methane conversion, gas yield, liquid yield, coke yield, and overall mass balance were measured and listed in Table 4. The composition of gas products is listed in Table 5, and the total acid number (TAN) and oxygen content data are listed in Table 6.

### Example 12

1 wt % Ir-1 wt % Ga-5 wt % Ce/TiO₂-anatase catalyst was prepared in the following manner. A homemade TiO₂, anatase support structure in powder form was used without further treatment. The metal salts x moles of gallium (III) nitrate hydrate (Ga(NO₃)₃·xH₂O) and y moles of cerium (III) nitrate hexahydrate (Ce(NO₃)₃·6H₂O), as well as 1.5(x+y) moles of citric acid powder, was dissolved in deionized water to form a precursor solution. The TiO₂ support was then mixed with the precursor solution, followed by stirring at 80° C. until most of the water was evaporated. The obtained wet powder was first dried in an oven at 110° C. overnight, followed by calcination at 450° C. with the temperature climbing at a rate of 0.5° C./min in a muffle furnace under an air atmosphere for 4 hours. The calcined powder (Ga—Ce/TiO₂-anatase) was then added to an aqueous solution containing IrCl₃ and urea at the following ratio: 1.06 g of Ga—Ce/TiO₂, 1.04 10⁻³ M IrCl₃, 1.04 M urea, and 50 mL water. The resultant suspension was stirred at 80° C. for 16 hours to achieve Ir loading. The resultant powder was separated by a centrifuge and washed with deionized water, the operation was repeated 4 times. The obtained wet powder was first dried in an oven at 110° C. overnight, followed by calcination at 450° C. with the temperature climbing at a rate of 10° C./min in a muffle furnace under an air atmosphere for 4 hours. The catalyst is denoted as 1% Ir-1% Ga-5% Ce/TiO₂-A.

Bio-crude was pumped into a fixed bed reactor at a rate of 0.09 mL/min. 5 mL Ir—Ga—Ce/TiO₂-anatase catalyst was loaded onto the reactor. The reaction was carried out under 400° C. and a gas atmosphere of 2.7 MPa H₂ and 0.3 MPa N₂. The gas feeding rate was 100 sccm and the reaction lasted 3 hours. The outlet gas was directly connected to a micro-GC instrument for further analysis. The liquid product was collected in a stainless steel container cooled at −4° C. by a chiller. The solid product was collected together with the spent catalyst after the reaction, and quantified by TGA analysis. Methane conversion, gas yield, liquid yield, coke yield, and overall mass balance were measured and listed in Table 4. The composition of gas products is listed in Table 5, and the total acid number (TAN) and oxygen content data are listed in Table 6.

### Example 13

1 wt % Ir-1 wt % Ga-5 wt % Ce/TiO₂-rutile catalyst was prepared in the following manner. A TiO₂ (Titanium (IV) oxide, rutile, powder, <5 μm) support structure in powder form obtained from Sigma-Aldrich was used without further treatment. The metal salts x moles of gallium (III) nitrate hydrate (Ga(NO₃)₃·xH₂O) and y moles of cerium (III) nitrate hexahydrate (Ce(NO₃)₃·6H₂O), as well as 1.5(x+y) moles of citric acid powder, was dissolved in deionized water to form a precursor solution. The TiO₂ support was then mixed with the precursor solution, followed by stirring at 80° C. until most of the water was evaporated. The obtained wet powder was first dried in an oven at 110° C. overnight, followed by calcination at 450° C. with the temperature climbing at a rate of 0.5° C./min in a muffle furnace under an air atmosphere for 4 hours. The calcined powder (Ga—Ce/TiO₂-rutile) was then added to an aqueous solution containing IrCl₃ and urea at the following ratio: 1.06 g of Ga—Ce/TiO₂-rutile, 1.04 10-3 M IrCl₃, 1.04 M urea, and 50 mL water. The resultant suspension was stirred at 80° C. for 16 hours to achieve Ir loading. The resultant powder was separated by a centrifuge and washed with deionized water, the operation was repeated 4 times. The obtained wet powder was first dried in an oven at 110° C. overnight, followed by calcination at 450° C. with the temperature climbing at a rate of 10° C./min in a muffle furnace under an air atmosphere for 4 hours. The catalyst is denoted as 1% Ir-1% Ga-5% Ce/TiO₂—R.

Bio-crude was pumped into a fixed bed reactor at a rate of 0.09 mL/min. 5 mL Ir—Ga—Ce/TiO₂-rutile catalyst was loaded onto the reactor. The reaction was carried out under 400° C. and a gas atmosphere of 3.0 MPa N₂. The gas feeding rate was 100 sccm and the reaction lasted 3 hours. The outlet gas was directly connected to a micro-GC instrument for further analysis. The liquid product was collected in a stainless steel container cooled at −4° C. by a chiller. The solid product was collected together with the spent catalyst after the reaction, and quantified by TGA analysis. Methane conversion, gas yield, liquid yield, coke yield, and overall mass balance were measured and listed in Table 4. The composition of gas products is listed in Table 5, and the total acid number (TAN) and oxygen content data are listed in Table 6.

TABLE 4
Overall analysis results in Examples 11-13.

| | | | Gas | Liquid | Coke | Overall |
|---|---|---|---|---|---|---|
| | | | yield | yield | yield | mass |
| Ex- | | | (wt | (wt | (wt | balance |
| ample | Catalyst | Gas | %) | %) | %) | (%) |
| 11 | 1% Ir-1% Ga- | 3.0 MPa N₂ | 2.09 | 95.25 | 2.02 | 99.36 |
| | 5% Ce/TiO₂-A | | | | | |
| 12 | 1% Ir-1% Ga- | 2.7 MPa H₂, | 0.25 | 98.84 | 0.33 | 99.42 |
| | 5% Ce/TiO₂-A | 0.3 MPa N₂ | | | | |
| 13 | 1% Ir-1% Ga- | 3.0 MPa N₂ | 2.02 | 95.23 | 1.85 | 99.10 |
| | 5% Ce/TiO₂-R | | | | | |

TABLE 5
Gas analysis of products in Example 11-13.

| | | | H₂ | C2—C5 | CO₂ |
|---|---|---|---|---|---|
| | | | yield | yield | yield |
| Example | Catalyst | Gas | (wt %) | (wt %) | (wt %) |
| 11 | 1% Ir-1% Ga- | 3.0 MPa N₂ | 0.56 | 0.24 | 1.77 |
| | 5% Ce/TiO₂-A | | | | |
| 12 | 1% Ir-1% Ga- | 2.7 MPa H₂, | / | 0.12 | 0.29 |
| | 5% Ce/TiO₂-A | 0.3 MPa N₂ | | | |
| 13 | 1% Ir-1% Ga- | 3.0 MPa N₂ | 0.23 | 0.22 | 1.57 |
| | 5% Ce/TiO₂-R | | | | |

TABLE 6
Oxygen content & TAN data in Example 11-13.

| | | | Oxygen | TAN |
|---|---|---|---|---|
| | | | content | (mg KOH/g |
| Example | Catalyst | Gas | (wt %) | sample) |
| 11 | 1% Ir-1% Ga- | 3.0 MPa N₂ | 0.22 | 0.31 |
| | 5% Ce/TiO₂-A | | | |
| 12 | 1% Ir-1% Ga- | 2.7 MPa H₂, | Trace | 0.20 |
| | 5% Ce/TiO₂-A | 0.3 MPa N₂ | | |
| 13 | 1% Ir-1% Ga- | 3.0 MPa N₂ | 0.25 | 0.30 |
| | 5% Ce/TiO₂-R | | | |

It is evident that the oxygen-containing species can be effectively removed from the bio-crude feedstock along with a significant reduction of TAN with the presence of methane environment and catalyst structure disclosed in the embodiments. Therefore, the embodiments as described herein provide an alternative way for an oil deoxygenation process, which is innovative and transformational in the biofuel industry.

While the invention has been described in detail and with reference to specific embodiments thereof, it will be apparent to one skilled in the art that various changes and modifications can be made therein without departing from the spirit and scope thereof.

Thus, it is intended that the present invention covers the modifications and variations of this invention provided they come within the scope of the appended claims and their equivalents.

What is claimed:

1. A method for deoxygenation of an oxygen-containing hydrocarbon product, the method comprising:
introducing a methane-containing gas into a reactor;
introducing an oxygen-containing hydrocarbon feedstock into the reactor; and
reacting the oxygen-containing hydrocarbon feedstock within the reactor in the presence of the methane-containing gas and a catalyst structure to form a hydrocarbon product having an oxygen content that is less than the oxygen content of the oxygen-containing hydrocarbon feedstock;
wherein:
the catalyst structure comprises porous support and one or more metals loaded within the porous support;
the porous support comprises one or more of an aluminum oxide material, an aluminosilicate material, an anatase material, a rutile material, a titanium silicalite material; and
one or more metals comprises one or more of Ir, Ga, and Ce.

2. The method of claim 1, wherein the oxygen-containing hydrocarbon feedstock comprises a bio-crude liquid having an oxygen content of at least 2 wt %.

3. The method of claim 1, wherein the hydrocarbon product has an oxygen content of less than 2 wt %, or no greater than 1 wt %, or no greater than 0.8 wt %, or no greater than 0.7 wt %, or no greater than 0.5 wt %.

4. The method of claim 1, wherein each metal is loaded within the porous support in an amount from about 0.1 wt % to about 10 wt % based upon the total weight of the catalyst structure.

5. The method of claim 1, wherein at least one metal is loaded within the porous support in the form of a nitrate or a chloride.

6. The method of claim 1, wherein the following metals are loaded within the porous support of the catalyst structure at the following weight percentages based upon the total weight of the catalyst structure: 1 wt % Ir, and 1 wt % Ga.

7. The method of claim 6, wherein Ce is further loaded within the porous support of the catalyst structure in an amount of 1 wt %.

8. The method of claim 6, wherein Ce is further loaded within the porous support of the catalyst structure in an amount of 5 wt %.

9. The method of claim 6, wherein Ce is further loaded within the porous support of the catalyst structure in an amount of 10 wt %.

10. The method of claim 1, wherein the following metals are loaded within the porous support of the catalyst structure at the following weight percentages based upon the total weight of the catalyst structure: 1 wt % Ir, and 5 wt % Ce.

11. The method of claim 1, wherein the following metals are loaded within the porous support of the catalyst structure at the following weight percentages based upon the total weight of the catalyst structure: 1 wt % Ga, and 5 wt % Ce.

12. The method of claim 1, wherein the following metals are loaded within the porous support of the catalyst structure at the following weight percentages based upon the total weight of the catalyst structure: 0.1 wt % Ir, 1 wt % Ga, and 5 wt % Ce.

13. The method of claim 1, wherein the catalyst structure is formed of porous support comprising an anatase material having the following metals loaded in the porous support structure at the following weight percentages based upon the total weight of the catalyst structure: 1 wt % Ir, 1 wt % Ga, and 5 wt % Ce.

14. The method of claim 1, wherein the catalyst structure comprises a plurality of granules.

15. The method of claim 1, wherein the reaction of the oxygen-containing hydrocarbon feedstock within the reactor occurs at a reaction temperature within a range from about 350° C. to about 450° C. and reaction pressure within a range from about 1 atm to about 50 atm.

16. The method of claim 15, wherein the reactor comprises a continuous reactor, and then reacting the oxygen-containing hydrocarbon feedstock within the reactor occurs at a liquid hourly space velocity (LHSV) of the feedstock being within a range from about 0.1 h⁻¹ to about 10 h⁻¹.

17. A catalyst structure comprising:
a porous support and one or more metals loaded within the porous support, wherein:
the porous support comprises one or more of an aluminum oxide material, an aluminosilicate material, an anatase material, a rutile material, a titanium silicalite material; and
one or more metals comprises one or more of Ir, Ga, and Ce.

18. The catalyst structure of claim 17, wherein each metal is loaded within the porous support in an amount from about 0.1 wt % to about 10 wt % based upon the total weight of the catalyst structure.

19. A method of forming the catalyst structure of claim 17, the method comprising:
dissolving two or more metal salts in water to form a metal precursor solution;
loading the metal precursor solution into the porous support;
drying the porous support loaded with the metal precursor for at least 2 hours at a temperature from about 80° C. to about 120° C.; and
calcining the dried porous support loaded with metal precursor at a temperature ranging from about 450° C. to about 700° C. and at a heating rate ranging from about 0.5° C./min to about 20° C./min.
